(19) United States
(12) Patent Application Publication
(10) Pub. No.: US 2004/0048892 A1
(43) Pub. Date: Mar. 11, 2004

(54) Treatment of diseases with adamantane derivatives

(76) Inventors: J. Peter Ruppersberg, Tuebingen (DE); Bernd Fakler, Malterdingen (DE)

Correspondence Address: NATH & ASSOCIATES, 1030 15th STREET, 6TH FLOOR, WASHINGTON, DC, 20005, US

(21) Appl. No.: 10/380,845

(22) PCT Filed: Sep. 21, 2001
(86) PCT No.: PCT/EP01/10927

(30) Foreign Application Priority Data
Sep. 21, 2000 (EP) 00120660.6
Sep. 29, 2000 (EP) 00121508.6

(51) Int. Cl. A61K 31/445; A61K 31/13; A61K 31/40

(52) U.S. Cl. 514/319; 514/408; 514/662

(57) ABSTRACT

The invention relates to the use of substances for the manufacturing of a pharmaceutical composition or medicament for the treatment of disturbances or illnesses which are linked to malfunction of an ionotropic acetylcholine receptor and/or a calcium-activated potassium channel functionally associated with said acetylcholine receptor, wherein said substance is at least one adamantane derivative.

The invention relates to the use of substances for the manufacturing of a pharmaceutical composition or medicament for the treatment of disturbances or illnesses which are linked to malfunction of an ionotropic acetylcholine receptor. Furthermore, the invention relates to the use of substances for the manufacturing of a pharmaceutical composition or a medicament for the treatment of disturbances or illnesses which are linked to malfunction of a calcium-activated potassium channel functionally associated with that ionotropic acetylcholine receptor.

The acetylcholine receptor is a ligand-gate cation channel. It is a glycoprotein composed of different types of transmembrane polypeptides. Each of those polypeptides is coded by a separate gene, although they all show strong similarities in their amino acid sequences, implying that their genes have evolved from a single gene.

Like the voltage-gated Na⁺-channel, the acetylcholine-gated channel has a number of discrete alternative conformations and in the presence of the ligand jumps randomly from one to another, switching abruptly between closed and opened states. Once it has bound acetylcholine and made the transition to the opened stage, it remains open for a randomly variable length of time, averaging about one millisecond. Greatly prorogated exposure to acetylcholine causes the channel to enter a desensitized state. In the open conformation., the channel has a lumen at its extracellular end, narrowing towards cell interior to a small pore. The charge distribution in the channel wall is such that negative ions are excluded, while any positive ion can pass through. The normal traffic consists of Na⁺ and K⁺, together with some Ca²⁺. Since there is little selectivity among cations, their relative contributions to the current through the channel depends mainly on their concentrations and on the electrochemical driving forces. Opening of the acetylcholine receptor channel leads to a large net influx of positive ions, causing membrane depolarization. This membrane depolarization causes a signal transmission from one membrane to another. A malfunction of this receptor can cause several diseases, and a malfunction of the acetylcholine receptor in the brain is e.g. discussed as reason for Alzheimer disease.

It is the object of the invention to convert new scientific insights into a new strategy for the treatment of important diseases. This object is solved by the subject-matter of claims 1 and 8. Preferred embodiments are given in the dependent claims 2 to 7 and 9 to 15. The wording of all these claims is hereby incorporated into the content of the description by reference.

Surprisingly it was found, that the acetylcholine receptor in the inner ear comprises of two subunits, namely alpha9 and alpha10. These subunits can be blocked by memantine, an adamantane derivative known for treatment of tinnitus associated with a so-called positive recruitment and/or a reduction or failure of otoacoustic emissions (EP 0759 295). Furthermore, it was found, that this acetylcholine receptor in the inner ear is functionally associated with a calcium-activated potassium channel, namely of the SK subtype. These findings make it possible to use adamantane derivatives for the treatment of disturbances that are linked to malfunction of this acetylcholine receptor and/or this calcium-activated potassium channel.

According to the invention, at feast one substance is used for the manufacturing of a pharmaceutical composition or a medicament for the treatment of disturbances or illnesses which are linked to malfunction of at least one ionotropic acetylcholine receptor, wherein that substance is an adamantane derivative and wherein that disturbance or illness is not a tinnitus associated with a so-called positive recruitment and/or a reduction or failure of otoacoustic emissions. According to the present invention, the acetylcholine receptor preferably comprises at least one so-called alpha9-subunit and alternatively or in addition it comprises at least one so-called alpha10-subunit. The substance which is used for the manufacturing of a pharmaceutical composition or medicament can be used in the form of its pharmaceutical acceptable salts and/or optionally together with a pharmaceutically carrier.

According to the invention, the above mentioned acetylcholine receptor is preferably functionally associated with at least one calcium-activated potassium channel. This calcium-activated potassium channel can be characterized in that it is of SK (small conductance) subtype, wherein preferably that SK potassium channel is of the SK2 subtype.

In one preferred embodiment of this invention the adamantane derivatives which are used for the manufacturing of a pharmaceutical composition or a medicament are of the formula

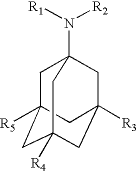

where R₁ and R₂

are the same or different, and include hydrogen or straight or branched chain alkyl groups having 1 to 6 C atoms, or

together with the N atom present a heterocyclic group having 5 or 6 ring atoms,

where R₃ and R₄ are the same or different, and include hydrogen, straight or branched chain alkyl groups having 1 to 6 C atoms, cycloalkyl groups having 5 to 6 C atoms, or phenyl, and

where R₅ is hydrogen or a straight or branched chain alkyl group having 1 to 6 C atoms.

In a preferred embodiment of this invention, this adamantane derivative is 3.5-dimethyl-1-adamantanamine or the hydrochloride thereof.

In other preferred embodiments according to the invention, the use of the substance is claimed for the manufacturing of a pharmaceutical composition or a medicament for the treatment of disturbances or illnesses linked to malfunction of at least one calcium-activated potassium channel functionally associated with a ionotropic acetylcholine receptor, wherein that substance is an adamantane derivative and wherein that disturbance or illness is not a tinnitus associated with a so-called positive recruitment and/or a reduction or failure of otoacoustic emissions.

According to the invention, this acetylcholine receptor which is functionally linked with a calcium-activated potassium channel preferably comprises at least one so-called alpha9-subunit and alternatively or in addition it comprises at least one so-called alpha 10-subunit.

In a preferred embodiment according to the invention, the calcium-activated potassium channel is of the SK subtype, wherein preferably that SK potassium channel is of the SK2 subtype.

According to the invention, the adamantane derivative which can be used for the manufacturing of a pharmaceutical composition or a medicament for the treatment of disturbances or illnesses that are linked to malfunctions of the above mentioned potassium channel are of the formula

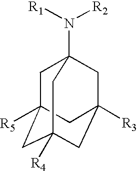

where R₁ and R₂

are the same or different, and include hydrogen or straight or branched chain alkyl groups having 1 to 6 C atoms, or

together with the N atom present a heterocyclic group having 5 or 6 ring atoms,

where R₃ and R4 are the same or different, and include hydrogen, straight or branched chain alkyl groups having 1 to 6 C atoms, cycloalkyl groups having 5 to 6 C atoms, or phenyl, and

where R₅ is hydrogen or a straight or branched chain alkyl group having 1 to 6 C atoms.

Preferably this adamantane derivative is 3.5-dimethyl-1-adamantanamine or the hydrochloride thereof.

The invention can be useful for treatment of all kinds of disturbances or illnesses which are linked with malfunction of an ionotropic acetylcholine receptor as defined above or the calcium-activated potassium channel which is functionally associated with the above mentioned acetylcholine receptor and which is also defined above. One preferred disturbance which can be treated by the invention is leukaemia, but also other known or up to now unknown diseases which are linked to these malfunctions defined above can be treated.

Another preferred illness to be treated according the invention is deafness, especially inner ear deafness. It is known that outer hair cells comprise an acetylcholine receptor and that alpha9- and alpha10-subunits of this receptor are expressed in outer hair cells. As this receptor is permeable for Ca²⁺-ions, a toxic effect of such Ca²⁺-ions is possible, resulting in a temporary or permanent impairment of the outer hair cells. Such impairment can be amended or even cured by the inventive use. In a preferred embodiment according to the invention the inner ear deafness is a acute one, prior to the manifestation of a permanent state of inner ear deafness.

Finally, according to the invention it is possible to select the administration form of the substance. This form can be adapted to the age, sex or other characteristics of the patient, the severity of the disturbances or illnesses or other parameters. Conventional additives can be present. The dosage can be freely selected as a function of the clinical picture and the condition of the patient.

The described features and further features of the invention can be gathered from the following description of preferred embodiments in conjunction with the subclaims. The individual features can be implemented separately or in the form of subcombinations.

### Materials and Methods

### Patch-clamp Recordings on Outer Hair Cells

The apical turn of the organ of Corti was dissected from cochleae of three to six week old Wistar rats as described previously (Oliver et al., 1999, J Physiol (Lond) 519, 791-800; Oliver et al., 2000, Neuron 26, 595-601). The preparation was performed in a solution containing (in mM) 144 NaCl, 5.8 KCl, 0.1 CaCl₂, 2.1 MgCl₂, 10 HEPES, 0.7 Na₂HPO₄, 5.6 glucose, pH adjusted to 7.3 with NaOH. For recordings, OHCs located between half and one turn from the apex of the cochlea were chosen. If necessary, supporting cells were removed with gentle suction from a cleaning pipette carefully avoiding mechanical disturbance of the efferent nerve terminals.

Whole-cell patch-clamp recordings were performed with an Axopatch 200B amplifier (Axon Instruments, Foster City, Calif.) at room temperature (22-25° C.). Electrodes were pulled from quartz glass, had resistances of 2-3 MΩ and were filled with intracellular solution (in mM): 135 KCl, 3.5 MgCl₂, 0.1 CaCl₂, 5 EGTA, 5 HEPES, 2.5 Na₂ATP. For one series of experiments, an intracellular solution containing the Ca²⁺-chelator BAPTA was used (in mM): 120 KCl, 3.5 MgCl₂, 10 BAPTA, 5 HEPES, 2.5 Na₂ATP. The pH of both solutions was adjusted to pH 7.3 with KOH. Membrane potential was corrected for the electrode junction potential (−4 mV). Whole-cell series resistance ranged from 4 to 9 MΩ and was not compensated. Currents were filtered at 1 kHz and sampled at 5 kHz. The specimen were continuously superfused with extracellular solution (in mM: 144 NaCl, 5.8 KCl, 2 CaCl₂, 0.9 MgCl₂, 10 HEPES, 0.7 Na₂HPO₄, 5.6 glucose, pH adjusted to 7.3 with NaOH). Chemicals as well as depolarizing solutions were applied via a glass capillary (diameter approximately 80 μm) positioned close to the organ of Corti. For the depolarizing external solution KCl was substituted for an equal amount of NaCl to result in [K⁺]ₑₓ of 47 mM. Memantine and acetylcholine (both from Sigma) were added to the extracellular solution from aqueous stock solutions. To block the large OHC resting K⁺ current, I_{k,n′} 10 μM lino-pirdine (RBI) or 1-5 pM XE991 (obtained from DuPont) was added to the standard extracellular medium from stock solutions made with DMSO (final concentration<0.1 %) (Housley and Ashmore, 1992, J Physiol (Lond) 448, 73-98; Marcotti and Kros, 1999, J Physiol (Lond) 520, 653-660). XE991 is a M-current blocker (Wang et al., 1998, Science 282, 1890-1893) that inhibits I_{k,n} at submicromolar concentrations in a poorly reversible manner.

### Electrophysiology on Heterologously Expressed Channels

In vitro mRNA synthesis and oocyte injections were performed as previously described (Fakler et al., 1995, Cell 80, 149-154; Oliver et al., 2000, see above). The electrophysiology on oocytes was also as described previously (Oliver et al., 2000, see above). In detail, AChR subunits and SK2 channels were heterologously expressed in Xenopus oocytes. Oocytes were surgically removed from adult females and dissected manually. 4-5 day prior to electrophysiological recordings, Dumont stage VI oocytes were injected with about 50 ng RNA. For coexpression experiments, the total amount of RNA was kept constant and the different RNAs were coinjected in equal concentrations. Two-electrode voltage-clamp measurements were performed with a TurboTec 01C amplifier (npi, Tamm, Germany), using microelectrodes of 0.1 to 0.5 MΩ filled with 3 M KCl. Extracellular medium was CaNFR, containing (in mM): 120 KCl, 2.5 KCl, 2 CaCl₂, 10 HEPES, pH adjusted to 7.3 with NaOH. For experiments in the absence of extracellular Ca²⁺, the extracellular solution was MgNFR (in mM): 120 KCl, 2.5 KCl, 2 MgCl₂, 10 HEPES, pH adjusted to 7.3 with NaOH. ACh and memantine were added from stock solutions and applied through an application system, that allowed solution exchange with a time constant of ˜1s. Currents were filtered at 100 Hz and sampled at a frequency of 1 kHz. Dose-inhibition relations obtained from electrophysiological experiments were fitted to the empirical Hill equation,

$${I}_{norm} = \frac{1}{1 + {( \frac{c}{{IC}_{50}} )}^{{}^{n} H}} \qquad ( 1 )$$

where Iₙₒᵣₘ is the normalized current, c is the blocker concentration, IC₅₀ is the half-inhibitory concentration, and n_{H} is the Hill coefficient. Data analysis and fitting was performed with IgorPro (Wavemetrics, Lake Oswego, Oreg.) on a Macintosh PowerPC. Unless stated otherwise, data are presented as mean±standard deviation.

### Molecular Biology

The coding region of the rat α10 gene (GenBank Accession No. AF196344) was amplified from rat brain cDNA by PCR using 5′- and 3′-adapter-primers containing suitable restriction sites (GA-GACCCGGGAGCTCCACC, ATGGGGACAAGGAGCCACTACC and GAGTCTAGATTACAGGGCTTGCACCAGTACAATG). The amplified fragments were subcloned into the Xenopus oocyte expression vector pGEM-HE (gift of Dr. J. Tytgat), yielding pGEM-HE-nAChR-α10, verified by sequencing. Capped mRNAs for α9, α10 and SK2 were synthesized in vitro using the mMESSAGE mMACHINE kit (Ambion, Austin, Tex.). To detect α10 and α9 transcripts, PCR was performed using reverse transcribed RNA isolated from either OHCs (containing some Deiters cells) or supporting cells (Deiters and Hensen's cells) as template. Cells were collected from rat organs of Corti using suction glass micropipettes (diameter 10 μm). RNA was prepared from ˜100 cells of each fraction using the Qiagen RNeasy kit (Qiagen, Hilden, FRG) according to the manufacturers instructions. The oligonucleotides used as primers in the PCR reactions were chosen to span an intron in the human α9 and α10 genes to allow differentiation between products originating from cDNA and products originating from contaminating genomic DNA.

| | |
|---|---|
| α9 sense: | CGTCCTCATATCGTTCCTCGCTCCG, |
| α9 antisense: | TGGTAAGGGCTGTGGAGGCAGTGA; |
| α10 sense: | GCAGCCTACGTGTGCAACCTCCTGC, |
| α10 antisense: | AGGTGTCCCAGCAGGAGAACCCGAG. |

For each PCR reaction, RNA corresponding to ˜3 cells was used as template.

### FIGURE LEGENDS

FIG. 1: Memantine dose-responses determined for the inhibitory postsynaptic currents (IPSC) in outer hair cells (OHCs).

FIG. 2: (A) ACh (100 μM) induced inward currents in oocytes coding for α9 but not in oocytes coding for α10. (B) Detection of α9 and α10 mRNA in OHCs by RT-PCR. (C) Same experiment as in (A), but for coexpression of both subunits. (D) Current amplitudes from experiments as in (A) and (C) summarized for oocytes injected with RNA coding for a non-conducting SK2 channel mutant (control), α9, α10, and α9/α10.

FIG. 3: (A) Currents evoked by activation of α9/α10 nAChRs are dependent on external Ca²⁺. (B) Currents from homomeric α9 and heteromeric α9/α10 nAChRs recorded by application of 100 μM ACh in nominally Ca²⁺-free external solution. (C) Current amplitudes from oocytes expressing α9 and α9/α10, recorded as in FIG. 3B. (D) ACh-induced (100 μM) currents recorded from α9/α10 nAChRs in the presence of the memantine concentrations indicated. (E) Memantine dose-inhibition curves of α9/α10 nAChR. (F) I-v relations of α9/α10 nAChR recorded in the absence (black) and presence (gray trace) of memantine.

FIG. 4: (A) Application of 100 μM ACh for the time indicated to an oocyte coexpressing α9, α10 and SK2. (B) K⁺ current induced by ACh (100 μM) in an oocyte as in (A) was reversibly blocked by memantine at the concentrations indicated. (C) Memantine dose-response curve of inhibition of the nAChR-induced SK2 current.

FIG. 5: (A) Steady-state block of nAChR currents in OHCs by memantine. (B) Dose-inhibition relation for memantine block of nAChR currents (squares), recorded from 4 OHCs as in (A).

FIG. 6: Inhibition of α9/α10 heteromeric acetylcholine receptors expressed in Xenopus oocytes by memantine.

### EXPERIMENT 1

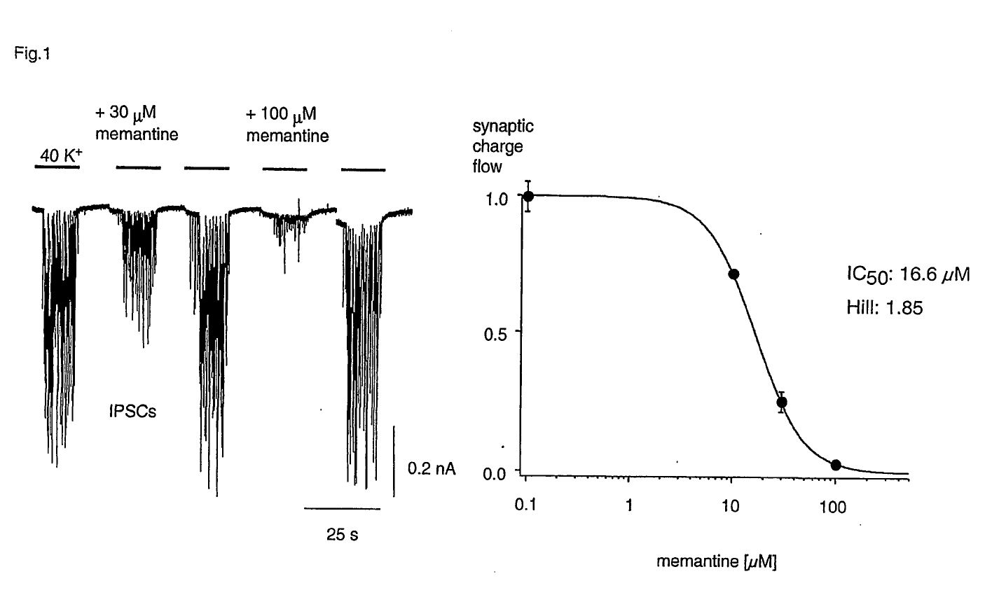

In FIG. 1, postsynaptic currents were recorded from OHCs in voltage-clamp experiments when cells were held at −64 mV and the whole Corti preparation was superfused with high extracellular K⁺ to depolarize the presynapse. Application of high K⁺ and toxins as indicated by horizontal bars: current and time scaling as indicated. The postsynaptic currents were reversibly inhibited by application of memantine, whereas there is a dose-dependent response of the OHCs (30 mM and 100 mM, respectively). This dose-dependent response determined for the IPSC in OHCs is illustrated in FIG. 1B. There it is shown, that in OHCs memantine has an IC₅₀ of approximately 16.6 μM.

### EXPERIMENT 2

α9 and α10 nAChR subunits are expressed in OHCs and coassemble to functional channels. In (A) ACh (100 μM) induced inward currents in oocytes injected with RNA coding for α9 (lower trace) but not in oocytes injected with RNA coding for α10 (upper trace). Holding potential was −80 mV. Fast downward spikes in the traces are artifacts resulting from switching between solutions. (B) Detection of α10 and α9 mRNA in OHCs by RT-PCR. Fragments of the expected length were amplified for α9 and α10 from OHCs (lanes 1, 2) but not from supporting cells (lane 3, data for α9 not shown). Controls were OHC-RNA without RT added (lane 4) and water (lane 5) as a template for PCR. (C) Same experiment as in (A), but for coexpression of both subunits. Note the different current scaling. The recording from (A) (α9) was added for comparison. (D) Current amplitudes from experiments as in (A) and (C) summarized for oocytes injected with RNA coding for a non-conducting SK2 channel mutant (control), α9, α10, and α9/α10 (values are mean±standard error of 5, 13, and 18 oocytes, respectively).

The nAChR of outer hair cells has been shown to contain the α9 subunit by a variety of methods including in-situ hybridization (Elgoyhen et al., 1994, see above; Morley et al., 1998, Brain Res Mol Brain Res 53, 78-87) single-cell RT-PCR (Glowatzki and Fuchs., 1995, Science 288, 2366-2368>, transgenic expression of green fluorescent protein controlled by the α9. promotor (Zuo et al., 1999, Proc Natl Acad Sci 96, 14100-14105), and inactivation of the α9 gene (Vetter et al., 1999, Neuron 23, 93-103). Homomeric α9 receptors yield remarkably small currents when heterologously expressed in Xenopus oocytes (Elgoyhen et al., 1994, see above), raising the possibility that an additional subunit is needed to yield the fully functional OHC receptor. However, OHCs lack any other of the known nAChR subtypes (Morley et al., 1998, see above). A GenBank search yielded a new subunit of the nAChR family (GenBank Accession No. AF196344), designated as α10.

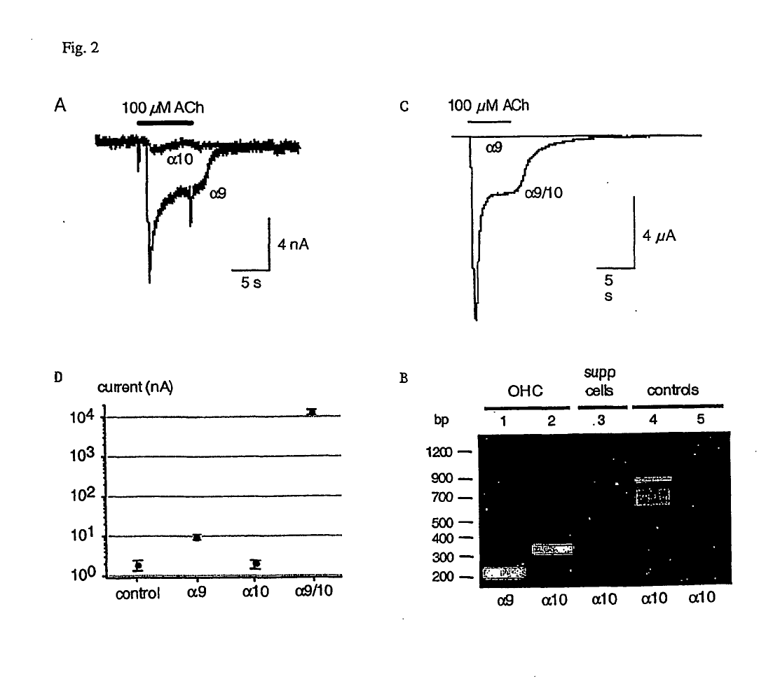

Therefore, to test whether the inhibition of the complex IPSCs resulted from block of Ca²⁺-entry via the nAChR and to test if α10 is a candidate subunit for the OHC receptor, OHC nAChR expressed heterologously in Xenopus oocytes were tested. However, applications of 100 μM ACh to oocytes injected with α9-specific RNA yielded very small currents (9.3+5.0 nA at −80 mV; FIG. 2A), consistent with previous reports (Elgoyhen et al., 1994, see above; Katz et al., 2000, Hear Res 141, 117-128). No currents exceeding background levels were observed with the rat homologue of the α10 subunit, a member of the nAChR family recently identified by Boulter and colleagues (GenBank Accession No. AF196344; FIG. 2A, D). As shown in FIG. 2B by RT-PCR on OHCs isolated from the rat organ of Corti (see Methods), α10 mRNA is indeed present in these sensory cells, while it was not detected in the supporting cell fraction, containing Hensen and Deiters cells. In a control experiment with RNA from OHCs that was not reverse transcribed, PCR amplified a fragment of ˜900 bp (FIG. 2B, lane 4) which most likely resulted from contamination with genomic DNA as the length of this fragment is in good agreement with the sequence defined by the primer pair in the human genome (BAC from chromosome 11; GenBank Acc.#AC060812). When both, α9 and α10, were coexpressed in oocytes, large inward currents with peak amplitudes of up to −35 μA (at −80 mV) were recorded upon application of ACh (FIG. 2C, D). Similar to α9-mediated currents, the timecourse was characterized by an initial transient declining to a smaller plateau of a variable aplitude with respect to the peak current.

The increase in current amplitude of more than 3 orders of magnitude (compared to homomeric α9 expression) together with the coexpression of α9 and α10 in OHCs suggest that heteromultimerization of both subunits is essential to give fully functional receptor channels. Moreover, the absence of any other of the known nAChR subunits (Morley et al., 1998, see above) strongly suggests that the OHC nAChR is a heteromer composed of α9 and α10 subunits.

### EXPERIMENT 3

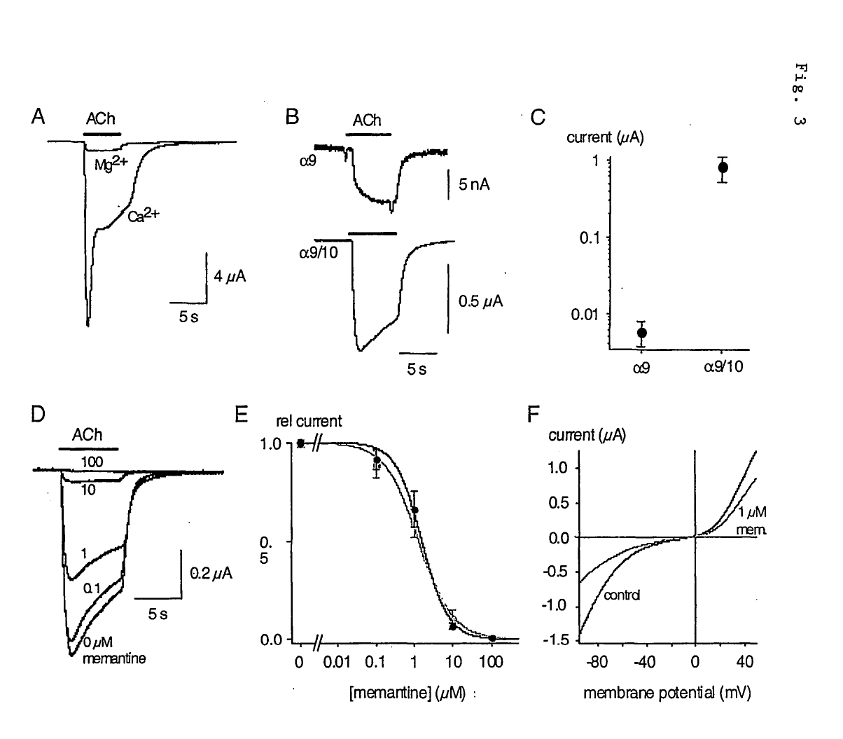

In (A) currents evoked by activation of α9/α10 nAChRs are dependent on external Ca²⁺. Traces show subsequent applications of 100 μM ACh to the same oocyte in CaNFR (Ca²⁺) and MgNFR (Mg²⁺) at −80 mV. (B) Currents from homomeric α9 (upper trace) and heteromeric α9/α10 nAChRs (lower trace), recorded by application of 100 μM ACh in nominally Ca²⁺-free external solution (MgNFR; −80 mV). (C) Current amplitudes from oocytes expressing α9 and α9/α10, recorded as in FIG. 3B (mean±standard error from 12 and 15 oocytes, respectively). (D) ACh-induced (100 μM) currents recorded from α9/α10 nAChRs in the presence of the memantine concentrations indicated. (E) Memantine dose-inhibition curves of α9/α10 nAChR. Peak currents were recorded as in (D) and normalized to the current amplitude after washout of the blocker. Continuous lines show fits of the Hill equation to data from 7 oocytes measured in MgNFR (solid) and CaNFR (grey), yielding IC₅₀ of 1.6 and 1.3 μM and nH of 1.2 and 1.0, respectively. (F) I-v relations of α9/α10 nAChR recorded in the absence (black) and presence (gray trace) of 1 μM memantine in response to 2 s voltage ramps from −100 to +50 mV. Currents were recorded in MgNFR and were leak-corrected by subtracting the response to the same voltage ramp preceding the application of 100 μm ACh.

A characteristic feature of homomeric α9 channels is their exceptionally high Ca²⁺-permeability (Jagger et al., 2000, J Physiol 527, 49-54; Katz et al., see above). This Ca²⁺-permeability is thought to be essential for the OHC nAChR, since it allows for a Ca²⁺ influx sufficiently high to effectively activate SK-type potassium channels. The oocyte expression system, however, is characterized by high endogenous expression levels of Ca²⁺-activated Cl⁻channels (Stühmer and Parekh, 1995, in Single-channel recording 2^{nd} edition (Neher E and Sakmann B eds) 341-356, Plenum Press, New York). Therefore, opening of Ca²⁺-permeable channels in an external medium containing Ca²⁺ leads to a coactivation of a Cl⁻ conductance. When external Ca²⁺ was substituted for Mg²⁺, currents induced by ACh application onto α9/α10 heteromeric channels were reduced by a factor of roughly 10 (FIG. 3A). Thus, a large fraction of the ACh-induced current measured in CaNFR was due to opening of Ca²⁺-activated Cl⁻ currents. This was also supported by the reversal potential of the current in CaNFR of about −25 mV (data not shown), close to the estimated Cl⁻ equilibrium potential in Xenopus oocytes (Stühmer and Parekh, 1995, see above). Consequently, α9/α10 heteromeric channels had a significant Ca²⁺-permeability, similar to what is known from homomeric α9 receptors. Application of ACh in the absence of extracellular Ca²⁺ allowed the recording of α9/α10 currents in isolation. Heteromeric channels yielded currents that were 100fold larger than currents recorded from α9 channels under the same conditions, confirming the large gain of receptor conductance by coexpression that was observed in the presence of external Ca²⁺(FIG. 3B, C). In the absence of Ca²⁺, α9/α10 also showed consistent kinetics characterized by slow desensitization on a time scale of seconds. Desensitization was not observed with α9 channels within the limits of the speed of solution exchange (FIG. 3B).

The action of memantine was tested on the isolated α9/α10 current in MgNFR. Memantine blocked this current in a completely reversible manner with an IC₅₀ of 1.6 μM and a Hill coefficient of 1.2 (FIG. 3D, E).

Channel block by memantine has been analyzed most extensively at NMDA-type glutamate receptors (Bresink et al., 1996, Br J Pharmacol 119, 195-204; Chen et al., 1992, J Nerosci 12, 4427-4436), where block of the open pore is strongly voltage-dependent. To address the issue of blocking mechanism and voltage dependence at α9/α10 channels, i-v relations were determined from voltage ramps in the absence and presence of memantine (FIG. 3F). In either case, i-v curves were highly nonlinear and showed considerable rectification at negative and positive potentials. The reversal potential was −6.4+1.0 mV (n=3), consistent with a non-specific cation channel. As shown in FIG. 3F, memantine block was observed over the whole voltage range tested. It increased by a factor of 1.6 from +50 mV to −80 mV and thus exhibited only mild voltage-dependence. We also measured the impact of memantine on the chloride current, activated secondarily in the presence of external Ca²⁺. Memantine inhibited the peak chloride current with an efficiency not significantly different from block of the isolated α9/α10 current (FIG. 3E).

### EXPERIMENT 4

(A) Application of 100 μM ACh for the time indicated to an oocyte coexpressing α9, α10, and SK2. Traces are subsequent recordings from the same oocyte at the holding potentials indicated. (B) K⁺ current induced by ACh (100 μM) in an oocyte as in (A) was reversibly blocked by memantine at the concentrations indicated. Holding potential was −30 mV. (C) Memantine dose-response curve of inhibition of the nAChR-induced SK2 current (n=5 oocytes). Peak outward currents were recorded at −30 mV and were normalized to the values preceding memantine application. Continuous line shows fit of the Hill: equation (IC₅₀=0.7 μM;. n_{H}=1.1).

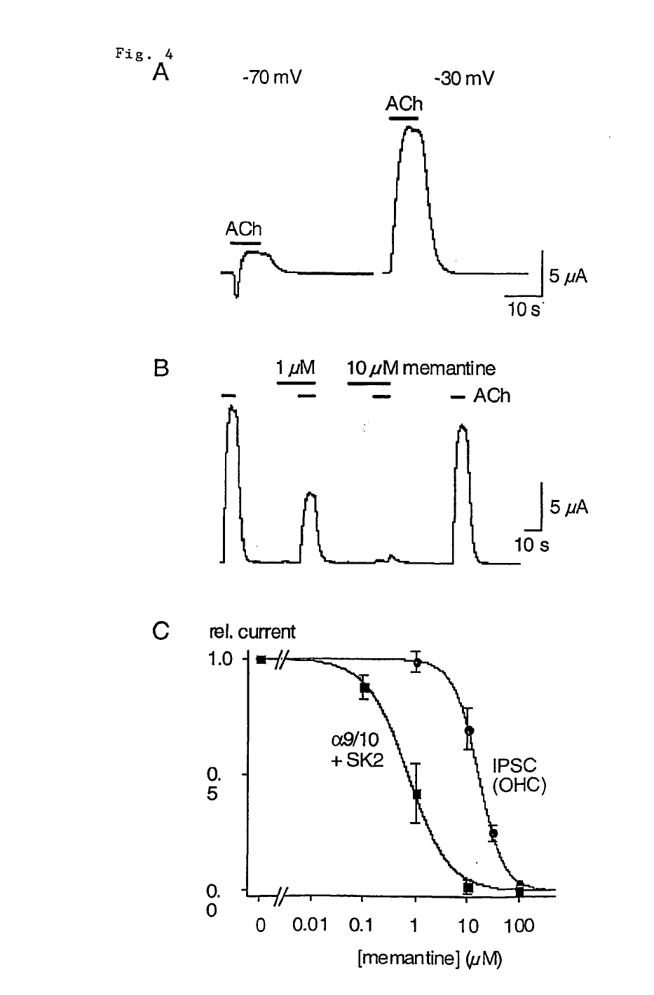

In hair cells, Ca²⁺ influx via nAChRs activates SK2 channels to give rise to IPSCs. FIG. 4 shows, that this activation cascade may be reconstituted in Xenopus oocytes by coexpression of the α9/α10 nAChR with SK2 channels. In oocytes expressing both channel species, application of ACh evoked a biphasic response at −70 mV. An initial inward current carried mainly by chloride (see above) was followed by an outward current due to the activation of SK2 channels (FIG. 4A). With the Cl⁻ driving force largely abolished and an increased driving force for K⁺ at a membrane potential of −30 mV, ACh induced a monophasic potassium outward current, similar to the response of isolated OHCs to ACh application (Blanchet et al., 1996, J Neurosci 16, 2574-2584; Evans, 1996, J Physiol (Lond) 491, 563-578). Memantine block of the SK2 current showed a dose-response relation that was characterized by an IC₅₀ of 0.7 μM and a Hill coefficient of 1.1 (FIG. 4B, C), very similar to the values obtained for the memantine block of α9/α10 receptors (see FIG. 3E). However, these values were considerably different from those obtained for memantine-induced inhibition of IPSCs in OHCs. Thus, inhibition of IPSCs required 10-fold higher concentrations of memantine than inhibition of SK2 currents in the oocyte system (FIG. 4C). This difference might suggest that the nAChR underlying the generation of IPSCs is less susceptible to memantine block than α9/α10 and may thus be indicative for a different subunit composition of OHC nAChRs.

### EXPERIMENT 5

(A) Steady-state block of nAChR currents in OHCs by memantine. Currents through nAChRs were recorded from an OHC at a holding potential of −94 mV. Inward currents induced by application of 200 μM ACh were reversibly blocked by the coapplication of memantine as indicated by horizontal bars. The intracellular solution contained 10 mM BAPTA to prevent activation of SK currents. (B) Dose-inhibition relation for memantine block of nAChR currents (squares), recorded from 4 OHCs as in (A). Steady-state amplitudes were normalized to the value recorded in the absence of memantine. The fit to the Hill equation (continuous line) yielded IC₅₀=1.1 μM and n_{H}=0.9. Dose-inhibition curves for memantine block of α9/α10 currents in oocytes (circles) and of IPSCs (triangles) are replotted for better comparison.

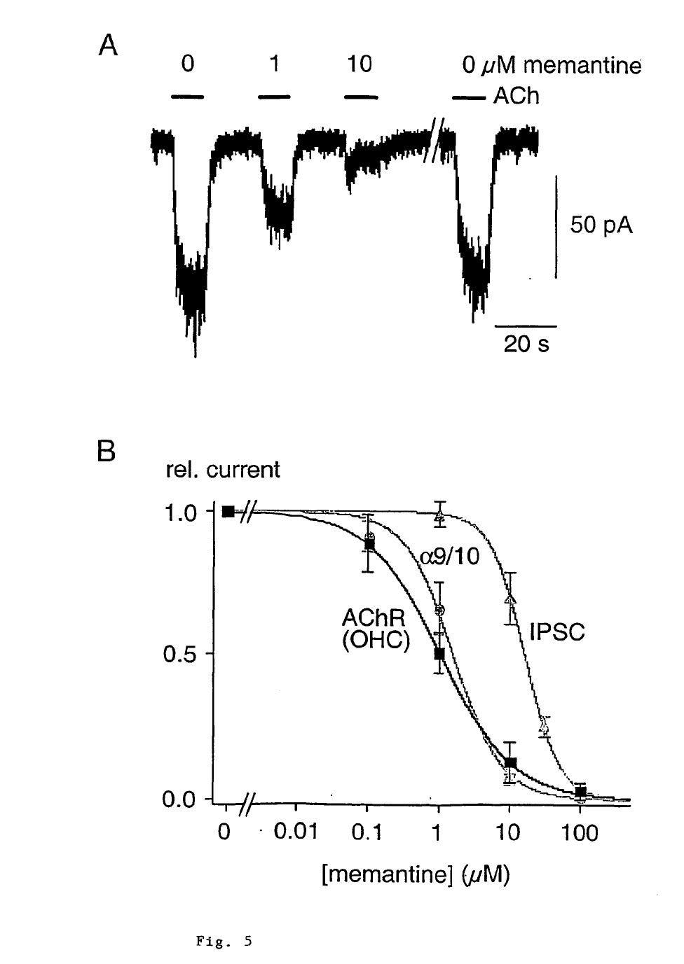

To test whether the difference in memantine block (see experiment 4) might suggest that the nAChR underlying the generation of IPSCs is less susceptible to memantine block than α9/α10 and may thus be indicative for a different subunit composition of OHC nAChRs, the effect of memantine on the nAChR of OHCs was directly examined. AChR currents can be measured in isolation by uncoupling SK channel activation from Ca²⁺ influx via the nAChR with the fast Ca²⁺-chelator BAPTA (Blanchet et al., 1996, see above; Fuchs and Murrow, 1992, J Neurosci 12, 800-809). Application of ACh to OHCs dialyzed with 10 mM BAPTA from the recording pipette induced inward currents of 110±39 pA at −94 mV (n=4). These currents were blocked by memantine with an IC₅₀ of 1.1 μM and a Hill coefficient of 0.9 (FIG. 5 A, B). This affinity is in close agreement with the values obtained for the block of α9/α10 receptors, and strongly suggests that the observed differences in blocking potency of memantine are not due to a different receptor but may instead result from the specific mechanism of coupling between nAChRs and SK channels in OHCs.

Heteromeric α9/α10 channels are reversibly blocked by memantine. This adamantane derivative is a well characterized open channel blocker of NMDA-type glutamate receptors with an IC₅₀ of around 1 μM (at −70 mV) and also blocks neuronal α4/β2 nAChRs with an IC₅₀ of 7 μM (at −100 mV) (Buisson and Bertrand, 1998, Mol Pharmacol 53, 555-563; Chen et al., 1992, see above; Parsons et al., 1993, Neuropharmacology 38, 735-767). Thus, the affinity for memantine of α9/α10 receptors (˜1μM, see FIGS. 3E, 5B) is considerably higher than of neuronal nAChRs and equals the affinity of NMDA receptors. For NMDA receptors, however, this high affinity is only observed at hyperpolarized potentials due to the pronounced voltage dependence of the block (electrical distance (δ) of about 1, Bresink et al., 1996, see above). In contrast, block of α9/α10 receptors was only weakly voltage-dependent; i.e. the channels exhibit high affinity block over the entire voltage range tested (−100 to +50 mV) with only a moderate increase of blocking efficacy at negative voltages. This differential blocking by memantine is paralleled by the pore-blocking observed with Mg²⁺. While Mg²⁺ completely occludes NMDA receptors under physiological conditions for extracellular Mg²⁺ and membrane potential (Burnashev et al., 1992; Mayer et al., 1984), α9/α10 receptors are virtually left unchanged by Mg²⁺ as indicated by a lack of current decrease at negative potentials (FIG. 3F). Accordingly, amplitude or timecourse of IPSCs in OHCs are not altered by removal of extracellular Mg²⁺.

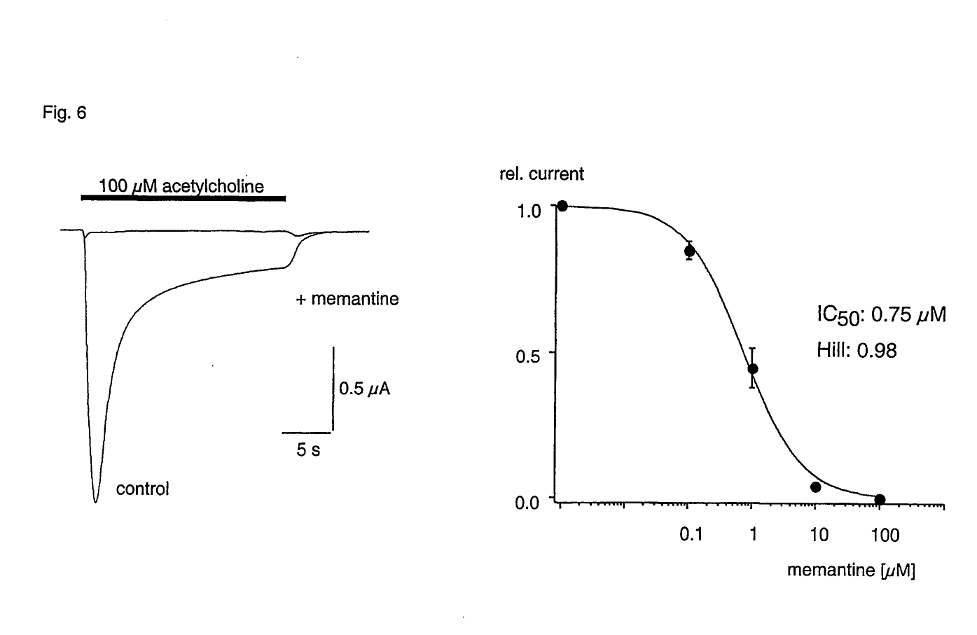

The sensitivity of OHC IPSCs for inhibition by memantine was an order of magnitude lower than the sensitivity of the receptor itself (FIG. 5B). This divergence may be explained by considering the blocking mechanism of memantine. As an open channel blocker, it will enter and occlude the pore only after the channel was opened by the ligand. AChRs underlying IPSCs open for some 20 ms, and complete activation of SK2 channels occurs within 10 ms after onset of the nAChR current (Oliver et al., 2000, see above). Thus, memantine must occlude the channel within milliseconds to effectively block IPSCs. Consequently, blocking efficacy will be determined more by the on-rate of the pore block than by the equilibrium affinity of the blocker that was measured with steady-state nAChR currents. Since the blocking time constant decreases with the blocker concentration, inhibition of IPSCs remains dependent on the concentration of memantine. Blocking the time constants on the order of seconds, as suggested by fast application of memantine at half-blocking concentrations to open a α4/β2 nAChRs (see FIG. 6B in Buisson and Bertrand, 1998, see above), indicate that blocking speed may indeed be limiting for the inhibition of fast IPSCs. In accordance with this consideration, the increased Hill coefficient of 2 with respect to n_{H}˜1 in all steady-state measurements suggests that inhibition of IPSCs is not determined by a simple steady-state pore block.

Additionally, non-linear coupling between α9/α10 receptors and SK2 channels may contribute to the observed difference in memantine block. Tight colocalization of both channels at the OHC synapse (Oliver et al., 2000) and/or enhancement of the buildup of high [Ca²⁺] domains by the subsynaptic cisterna acting as a diffusion barrier (Martin and Fuchs, 1992, Proc R Soc Lond B Biol Sci 250, 71-76) may enable complete activation of SK2 current by partially blocked nAChRs.

### EXPERIMENT 6

For further characterization of memantine treatment on the nAChR, the mRNA of alpha9/alpha10 subunits of the acetylcholine receptor were injected in oocytes of Xenopus laevis. After expression of that subunits, these subunits were investigated by treatment with memantine in voltage-clamp experiment. The cells were held at −30 mV. Current and time scaling are as indicated. As is shown in FIG. 6, similar results were obtained as in FIG. 1. In this experiment the polarization potential of the cells was measured after application of 100 μM acetylcholine in the presence or absence of memantine. Consistent with the results obtained in FIG. 1, the current responses at the acetylcholine receptor could be blocked by application with memantine. A halfmaximal inhibition occurs at concentrations of approximately 1 μM, as is illustrated in FIG. 6.

1. Use of a substance for the manufacturing of a pharmaceutical composition or medicament for the treatment of disturbances or illnesses linked to malfunction of at least one ionotropic acetylcholine receptor, wherein said substance is at least one adamantane derivative and wherein said disturbance or illness is not a tinnitus associated with a so-called positive recruitment and/or a reduction or failure of otoacoustic emissions.

2. Use according to claim 1, characterized in that said acetylcholine receptor comprises at least one alpha9-subunit.

3. Use according to claim 1 or claim 2, characterized in that said acetylcholine receptor comprises at least one alpha10-subunit.

4. Use according to one of the preceding claims, characterized in that said acetylcholine receptor is functionally associated with at least one calcium-activated potassium channel.

5. Use according to claim 4, characterized in that said calcium-activated potassium channel is of SK subtype, wherein preferably said SK potassium channel is SK2.

6. Use according to one of the preceding claims, characterized in that said adamantane derivatives are of the formula 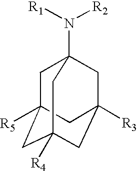
where R₁ and R₂
are the same or different, and include hydrogen or straight or branched chain alkyl groups having 1 to 6 C atoms, or
together with the N atom present a heterocyclic group having 5 or 6 ring atoms,
where R₃ and R₄ are the same or different, and include hydrogen, straight or branched chain alkyl groups having 1 to 6 C atoms, cycloalkyl groups having 5 to 6 C atoms, or phenyl, and
where R₅ is hydrogen or a straight or branched chain alkyl group having 1 to 6 C atoms.

7. Use according to claim 6, characterized in that said adamantane derivative is 3.5-dimethyl-1-adamantanamine or the hydrochloride thereof.

8. Use of a substance for the manufacturing of a pharmaceutical composition or a medicament for the treatment of disturbances or illnesses linked to malfunction of at least one calcium-activated potassium channel functionally associated with an ionotropic acetylcholine receptor, wherein said substance is at least one adamantane derivative and wherein said disturbance or illness is not a tinnitus associated with a so-called positive recruitment and/or a reduction or failure of otoacoustic emissions.

9. Use according to claim 8, characterized in that said acetylcholine receptor comprises at least one alpha9-subunit.

10. Use according to claim 8 or claim 9, characterized in that said acetylcholine receptor comprises at least one alpha10-subunit.

11. Use according to one of claims 8 to 10, characterized in that said calcium-activated potassium channel is of SK subtype, wherein preferably said SK potassium channel is SK2.

12. Use according to one of claims 8 to 11, characterized in that said adamantane derivatives are of the formula 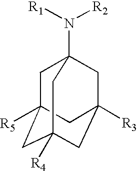
where R₁ and R₂
are the same or different, and include hydrogen or straight or branched chain alkyl groups having 1 to 6 C atoms, or
together with the N atom present a heterocyclic group having 5 or 6 ring atoms,
where R₃ and R₄ are the same or different, and include hydrogen, straight or branched chain alkyl groups having 1 to 6 C atoms, cycloalkyl groups having 5 to 6 C atoms, or phenyl, and
where R₅ is hydrogen or a straight or branched chain alkyl group having 1 to 6 C atoms.

13. Use according to claim 12, characterized in that said adamantane derivative is 3.5-dimethyl-adamantanamine or the hydrochloride thereof.

14. Use according to one of the preceding claims, characterized in that said disturbance or illness is leukaemia.

15. Use according to one of claims 1 to 13, characterized in that said disturbance or illness is deafness, especially inner ear deafness.
